(19) United States
(12) Patent Application Publication
(10) Pub. No.: US 2007/0098140 A1
(43) Pub. Date: May 3, 2007

(54) RADIOGRAPHIC IMAGING METHOD AND APPARATUS

(76) Inventors: Osamu Tsujii, Tochigi-ken (JP); Kazuhiro Matsumoto, Saitama-ken (JP)

Correspondence Address: COWAN LIEBOWITZ & LATMAN P.C.;JOHN J TORRENTE, 1133 AVE OF THE AMERICAS, NEW YORK, NY, 10036, US

(21) Appl. No.: 11/554,111

(22) Filed: Oct. 30, 2006

(30) Foreign Application Priority Data
Nov. 2, 2005 (JP) 2005-320007(PAT.)

(51) Int. Cl. H05G 1/60; A61B 6/00; G01N 23/00; G21K 1/12

(52) U.S. Cl. 378/20

(57) ABSTRACT

A radiographic imaging method controls a radiographic imaging apparatus comprises a support member which supports the subject while defining a backward direction going from the center of the rotation to the back of the subject. The method sets two rotational positions at which the backward direction and an irradiation direction going from the radiation source to the center of the rotation intersect approximately at right angles, as an irradiation start position and an irradiation end position for the radiations. The method designates one of the two rotational positions which is located in a range in which the angle formed by the backward direction and the irradiation direction decreases with the rotation is designated, as the irradiation start position.

### BACKGROUND OF THE INVENTION

1. Field of the Invention

The present invention relates to a radiographic image pickup apparatus which constructs images of radiation characteristic distributions in a subject using radiations in general, such as an X-ray CT scanner which uses X rays or other radiations for imaging.

2. Description of the Related Art

CT imaging includes full scan and half scan. The full scan involves collecting data in a range of 360 degrees while half scan involves collecting data in a range of 180 degrees plus a fan angle. One advantage of the half scan, which involves shorter acquisition time, is reduction of motion artifacts caused by movements of the body and movements of organs such as the heart.

CT imaging, which has a higher probability of detecting diseases than general radiography, has come into use for medical examination. However, it has the problem of increased X-ray dosage. Patient dosages are compared by calculating the effective dose based on doses absorbed by various organs as described in “A Research Report Supported by the Grant-in-Aid for Scientific Research on Priority Areas (C)(2), FY2002-2003: Development of a Measurement System for Organ Doses Resulting from Medical Exposure in Roentgenological Diagnosis” (Research Project No. 14580568) 2004, Takahiko Aoyama, et al. In relation to X-ray dosage, there are inventions which propose scanning methods capable of reducing radiation dosages received by X-ray technicians such as physicians.

For example, Japanese Patent Application Laid-Open No. 10-33525 discloses a method for collecting data by rotating an X-ray tube, where the method produces a zero dose of X-ray radiation in a predetermined angular range including an angle at which the X-ray tube, technician's hands, and subject are arranged in this order while producing a regular dose of X-ray radiation outside this rage. Also, Japanese Patent Application Laid-Open No. 11-290309 discloses a method which presets an IVR area for the technician to treat the subject. During one rotation of the X-ray tube, X-ray irradiation is stopped or decreased when the X-ray tube passes through an angular range corresponding to the preset IVR area and regular X-ray irradiation is performed when the X-ray tube is located outside the range. This greatly reduces the dosage received by the technician in the IVR area.

However, there is no discussion of a scanning method which can reduce the effective dose of the patient during CT imaging. This is because it is thought that the dosage does not depend on start and end angles of rotation in the case of a full scan and that the same X-ray dose, and thus the same X-ray dosage is required regardless of the scanning method—full scan or half scan.

### SUMMARY OF THE INVENTION

The present invention has been made in view of the above circumstances and has as its object reduction of the effective dose to which a patient is exposed during half scan CT imaging.

According to one aspect of the present invention there is provided a radiographic imaging apparatus which collects image data to perform CT half scan imaging, comprising: a rotation unit adapted to rotate a subject relative to a radiation source and radiation detector; an irradiation unit adapted to irradiate radiations from the radiation source to the subject rotated by the rotation unit; a support member, rotated with the subject, which has a face to support the back of the subject; and a control unit which controls the irradiation unit to execute the irradiation while the support member is turned to a side where the radiations enter.

According to another aspect of the present invention, there is provided a radiographic imaging apparatus which collects image data to perform CT half scan imaging, comprising: a rotation unit adapted to rotate a subject relative to a radiation source and radiation detector; an irradiation unit adapted to irradiate radiations from the radiation source to the subject rotated by the rotation unit; a support member, rotated with the subject, which has a face to support the front face of the subject; and a control unit which controls the irradiation unit to execute the irradiation while the support member is not turned to a side where the radiations enter.

Further features of the present invention will become apparent from the following description of exemplary embodiments with reference to the attached drawings.

### BRIEF DESCRIPTION OF THE DRAWINGS

FIG. 1 is a block diagram showing a radiographic imaging system according to an embodiment;

FIG. 2 is a system block diagram showing a configuration of a CT imaging apparatus according to the embodiment;

FIG. 3 is a flowchart illustrating operation of the CT imaging apparatus according to the embodiment;

FIG. 4 is a diagram illustrating a definition of an angle related to imaging operation; and

FIG. 5 is a diagram illustrating a preferred rotation start angle according to the embodiment.

### DESCRIPTION OF THE EMBODIMENTS

Preferred embodiments of the present invention will now be described in detail in accordance with the accompanying drawings.

The inventors have found experimentally that an effective dose varies with the start angle in the case of half scan. In the embodiment described below, in view of the variation in the effective dose, start position of a half scan in CT imaging is determined in such a way as to reduce the effective dose to the patient. Incidentally, in the following embodiment, the start position of a half scan is determined in such a way as to reduce the effective dose to the patient on a cone beam CT apparatus which takes X-ray CT images by rotating the patient. However, the present invention is not limited to this example, and may be applied to fan beam. CT or an apparatus which rotates a radiation source and detector with respect to a subject.

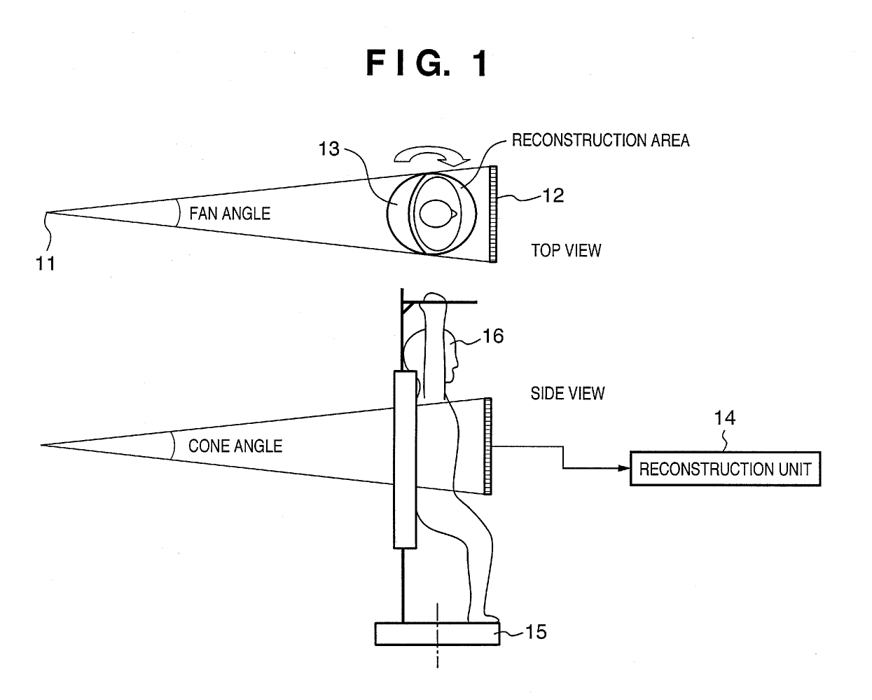

FIG. 1 is a diagram showing a configuration example of a CT imaging apparatus according to an embodiment. In this embodiment, X rays are used as radiation source. Under the conditions shown in FIG. 1, X-rays emitted from an X-ray generator 11 pass through a human body 16 as a subject and back rest 13 and reach a two-dimensional detector 12. The back rest 13 has a face to support the back of the subject. The two-dimensional detector 12 consists of a semiconductor sensor which, for example, has a resolution of 860×860 pixels and measures 43×43 cm in outside dimensions, with one pixel being 500×500 microns in size. Data acquired via the two-dimensional detector 12 is transferred to a reconstruction unit 14 to reconstruct images. A fan angle and cone angle are determined by geometric layout of the X-ray generator 11 (X-ray focus) and two-dimensional detector 12. According to this embodiment, which uses a square two-dimensional detector, the fan angle and cone angle are identical.

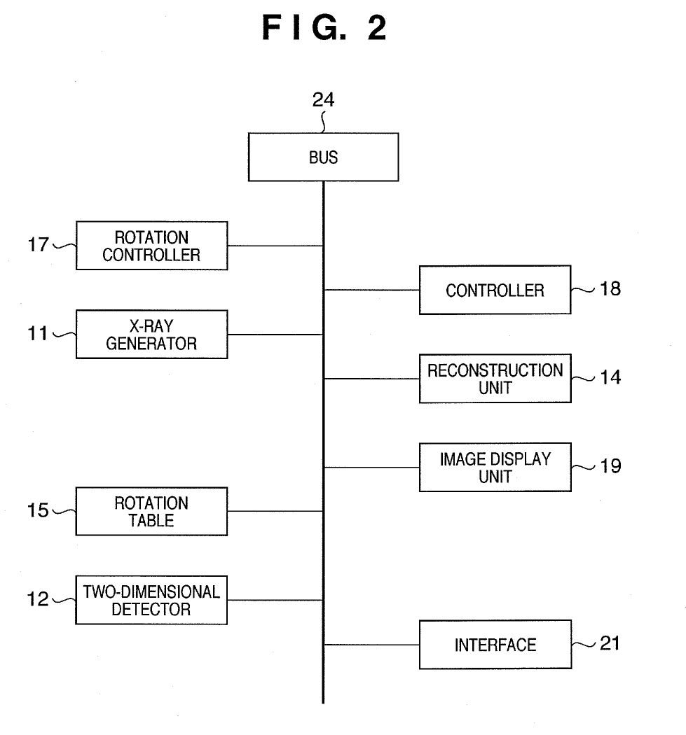

FIG. 2 is a system block diagram showing a configuration of the CT imaging apparatus according to this embodiment. The entire system is constructed around a computer system. The bus 24 is, for example, an internal bus of a computer. Control signals and data are transmitted and received via the bus 24. The controller 18 corresponds to a computer CPU. After a scan mode (full scan or half scan), rotation start position, rotational direction, and the like are input via an interface 21, a command to start imaging is issued. The controller 18 controls a rotation table 15, X-ray generator 11, and two-dimensional detector 12 based on input information about the scan mode (full scan or half scan), rotation start position, and rotational direction. The rotation controller 17 controls rotation of the rotation table 15 based on signals from a position sensor (not shown) and encoder (not shown) attached to the rotation table 15. Upon receiving ready-for-imaging signals from the rotation controller 17, two-dimensional detector 12, and X-ray generator 11, the controller 18 indicates (not shown) readiness for imaging, on the interface 21. When the operator gives a command to start imaging, the rotation table 15 with a human body 16 mounted thereon starts rotating on instructions from the controller 18.

During rotation of the rotation table 15, the controller 18 monitors angle information generated by the rotation controller 17, and thereby checks whether a predetermined fixed speed and angle have been reached. When the fixed speed and angle are reached, the controller 18 sends a signal to the X-ray generator 11 to start X-ray exposure.

If 1,000 views of projection data are collected per rotation of the rotation table 15 using an encoder which generates 25,000 pulses per one rotation, data is collected from the two-dimensional detector 12 every 25 pulses of an encoder signal. The rotation controller 17 counts the encoder pulses, outputs a timing signal to the two-dimensional detector 12 every 25 pulses, and detects the X-ray dose reaching each pixel of the two-dimensional detector 12. Although it is assumed in this embodiment that X-rays are generated continuously, this is not restrictive. Pulsed X-rays may be generated according to an integration interval of the two-dimensional detector 12 based on the encoder signal. The data obtained from the two-dimensional detector 12 is transferred sequentially to a reconstruction unit 14 via the bus 24. The data transfer continues until the rotation table 15 rotates a predetermined rotation angle and a predetermined number of views are collected. Upon completion of the X-ray exposure, the last projection data is collected. The collected projection data is reconstructed into 3D voxel data by the reconstruction unit 14.

A reconstruction process performed by the reconstruction unit 14 consists of preprocessing, filtering, and back projection processing. The preprocessing includes, an offset process, log transformation, gain correction, and defect correction. Generally, the Ramachandran function or Shepp-Logan function is used for filtering, and these functions are used in this embodiment as well. Filtered data is back-projected in back projection processing. Incidentally, the Feldkamp algorithm, for example, can be used for the processes from filtering to back projection. Once the back projection is completed and CT cross section images are reconstructed, the reconstructed cross sections are displayed on an image display unit 19.

The Feldkamp algorithm is used as a reconstruction algorithm, but this is not restrictive. References for reconstruction algorithms include “practical Cone-Beam Algorithm” (J. Opt. Soc. Am. Al. 612-619, 1984) presented by Feldkamp, Davis, and Kress.

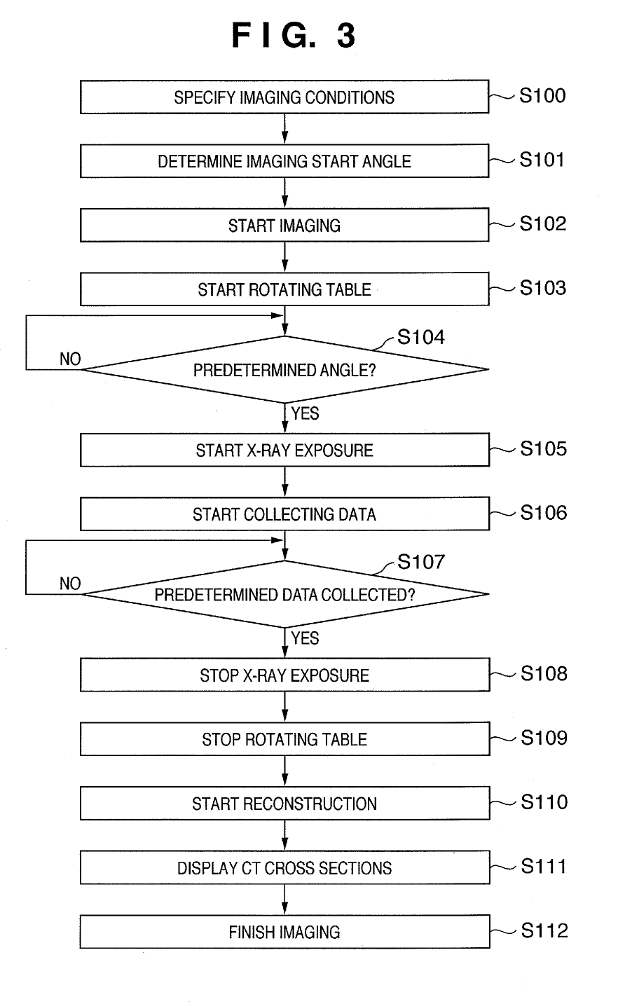

Next, operation of the CT imaging apparatus according to this embodiment will be described with reference to a flowchart in FIG. 3. In Step S100, imaging conditions are specified including a scan mode (full scan or half scan), rotation start position, rotational direction, rotation start angle, resolution of a transition angle, etc. X-ray exposure is started after passing the specified transition angle from the rotation start angle regardless of whether the scan mode is full scan or half scan. The transition angle is the angular difference between the rotation start angle and imaging start angle at which X-ray exposure is started. Thus, the angular difference includes a spin-up angle of the table.

In Step S101, the transition angle (imaging start angle) which optimizes an exposure dose (effective dose) is calculated based on the imaging conditions. Here, description will be given of how the transition angle passed until the start of X-ray exposure is determined according to the rotation start position and rotational direction when the scan mode is half scan. The full scan will not be discussed here because in the full scan mode, data is collected from all directions.

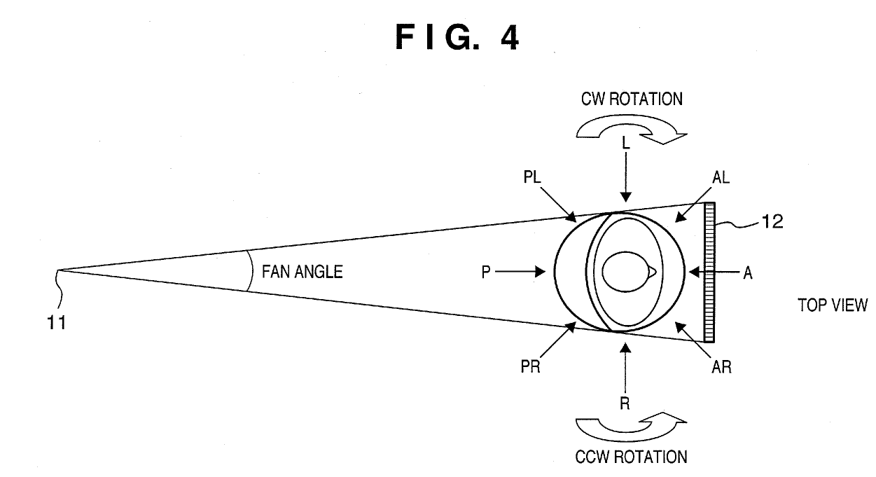

First, the rotation start angle will be defined with reference to FIG. 4. FIG. 4 shows an imaging geometric system as viewed from above. Regarding rotational directions of the rotation table with a human body mounted thereon, CW rotation (clockwise rotation) and CCW rotation (counterclockwise rotation) are defined, as indicated by arrows in the figure. The rotation start angle is defined with reference to the direction going from the X-ray generator 11 to the two-dimensional detector 12, i.e., the direction of X-ray irradiation axis. For example, if the rotation start position is located as shown in FIG. 4, the rotation start angle is “P.” Similarly, if rotation starts when the human body is facing the X-ray generator 11, the rotation start angle is “A.” Furthermore, if rotation starts when the left side of the human body is facing the X-ray generator 11, the rotation start angle is “L” and if rotation starts when the right side of the human body is facing the X-ray generator 11, the rotation start angle is “R.” Incidentally, if the resolution of the rotation start angle is set at 45 degrees, “PL,” “PR,” “AL,” and “AR” can be further defined as shown in FIG. 4.

Tables 1 and 2 show the transition angle determined from the rotation start angle and rotational direction to optimize the exposure dose(effective dose) in the half scan mode. They contain patterns (1) to (8) and patterns (9) to (16), respectively. In Table 1, the resolutions of the rotation start angle and transition angle are set at 90 degrees and in Table 2 the resolutions of the rotation start angle and transition angle are set at 45 degrees.

Although details of why the transition angle is determined will be described later, the transition angle in the tables is determined such that the imaging start angle will depend on the rotational direction and that the imaging start angle will be “L” in the case of CW rotation, and “R” in the case of CCW rotation. This causes the X-rays from the X-ray generator 11 to enter the human body mainly from the rear, making it possible to reduce the exposure dose (effective dose) because main organs such as the heart and stomach are located in the front part of the human body.

TABLE 1

| | Rotation | | Transition | Imaging |
|---|---|---|---|---|
| | Start | Rotation | Angle | Start |
| Pattern | Angle | Direction | (degree) | Angle |
| (1) | L | CW | 360 | L |
| (2) | L | CCW | 180 | R |
| (3) | A | CW | 90 | L |
| (4) | A | CCW | 90 | R |
| (5) | R | CW | 180 | L |
| (6) | R | CCW | 360 | R |
| (7) | P | CW | 270 | L |
| (8) | P | CCW | 270 | R |

TABLE 2

| | Rotation | | Transition | Imaging |
|---|---|---|---|---|
| | Start | Rotation | Angle | Start |
| Pattern | Angle | Direction | (degree) | Angle |
| (9) | AL | CW | 45 | L |
| (10) | AL | CCW | 135 | R |
| (11) | AR | CW | 135 | L |
| (12) | AR | CCW | 45 | R |
| (13) | PR | CW | 225 | L |
| (14) | PR | CCW | 315 | R |
| (15) | PL | CW | 315 | L |
| (16) | PL | CCW | 225 | R |

In Step S102, the operator gives a start-imaging command via the interface 21. Upon issuance of the start-imaging command, the rotation table 15 with a human body 16 mounted thereon starts rotating on instructions from the controller 18 in Step S103.

The controller 18 monitors the encoder signal (not shown) generated from the rotation table 15 and thereby checks whether a predetermined fixed speed and a data collection start position (imaging start angle) have been reached. When the predetermined fixed speed and the data collection start position are reached, the flow goes from Step S104 to Step S105. In Step S105, the controller 18 sends a signal to the X-ray generator 11 to start X-ray exposure. The encoder signal from the rotation table 15 is also used to determine the timing of integration of data. For example, if 1,000 views of projection data are collected per rotation of the rotation table 15 using an encoder which generates 25,000 pulses per rotation, data is collected from the two-dimensional detector 12 every 25 pulses of an encoder signal. In Step S106, the controller 18 counts the encoder pulses, generates an integration signal every 25 pulses, and detects the X-ray dose reaching the two-dimensional detector 12.

Although it is assumed in this embodiment that X-rays are generated continuously, this is not restrictive. Pulsed X-rays may be generated according to the integration interval of the two-dimensional detector 12 based on the encoder signal. Incidentally, the data from the two-dimensional detector 12 is transferred sequentially to the reconstruction unit 14 via the bus 24. The data transfer continues until the rotation table 15 rotates a predetermined rotation angle and a predetermined number of views are collected. When it is detected in Step S107 that the rotation table 15 has rotated the predetermined rotation angle and that the predetermined number of views have been collected, the processing goes to Step S108. In Step S108, the controller 18 instructs the X-ray generator to stop the X-ray exposure. Subsequently, the controller 18 decelerates the rotation table 15 to a stop in Step S109.

Upon completion of the X-ray exposure, the last projection data is transferred to the reconstruction unit 14. In Step S110, the controller 18 instructs the reconstruction unit 14 to perform reconstruction based on the collected projection data. Incidentally, the reconstruction unit 14 may perform reconstruction while collecting the projection data or start reconstruction after completion of all data collection. As described above, the process performed by the reconstruction unit 14 consists of preprocessing, filtering, and back projection processing. The preprocessing includes, an offset process, log transformation, gain correction, and defect correction. The Ramachandran function or Shepp-Logan function is used for filtering. Also, the Feldkamp algorithm is used for the processes from filtering to back projection. Once the back projection is completed and CT cross section images are reconstructed, the flow goes to Step S111, where the reconstructed cross sections are displayed on the image display unit 19. This concludes the imaging process according to this embodiment (Step S112).

Incidentally, there is demand to reduce not only the imaging time, but also a total imaging cycle including the time required to change the subject (human body 16) especially in the case of imaging for medical examination. The half scan imaging according to this embodiment is no exception.

In actual imaging operation, it is necessary to take into consideration:

a spin-up angle needed for the rotation table 15 to reach a predetermined speed at the imaging start angle at which imaging is started,

a spin-down angle needed for the rotation table 15 to, after imaging ends, decelerate from the predetermined speed until it stops at the imaging end angle, and

a fan angle.

If these angles are taken into consideration, the total rotation angle in one imaging flow exceeds 360 degrees by no less than 90 degrees in the case of patterns (1), (6), (7), (8), (13), (14), (15), and (16) in Tables 1 and 2 above.

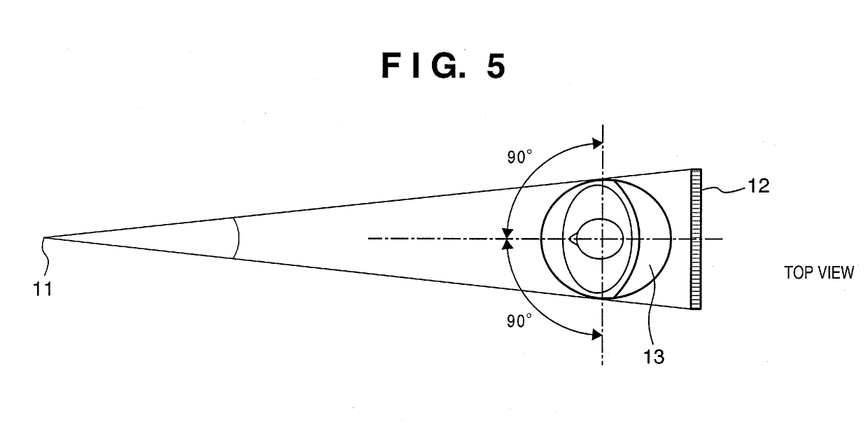

On the other hand, in the case of patterns (2), (3), (4), (5), (9), (10), (11), (12), as shown in FIG. 5, the rotation start angle is set within 90 degrees (inclusive) to the right and left from the reference position in which the human body 16 is facing the X-ray generator 11 along the X-ray irradiation axis. If such a rotation start angle is used, it is possible to keep the total rotation angle in one imaging flow generally within 360 degrees (inclusive). Furthermore, when such a rotation start angle is used, the two-dimensional detector 12 will never present an obstacle in front of the human body 16 unlike, for example, the rotation start angles in patterns (7), (8), (13), (14), (15), and (16). This makes it easier to change the human body 16 and secure it to a back rest, and thus provides rotation start angles suitable for the half scan mode.

As described above, the rotation start angle is set within 90 degrees (inclusive) to the right and left from the reference position in which the human body 16 is facing the X-ray generator 11 along the X-ray irradiation axis. This has the advantage of keeping the total rotation angle in one imaging flow within no more than 360 degrees, reducing the load on the human body caused by rotation as well as reducing the imaging cycle.

Furthermore, if the rotation start angle and rotation end angle of the rotation table 15 are made to coincide, it is no longer necessary to rotate the rotation table 15 between imaging cycles. This eliminates useless operations and loss of time, making it possible to further reduce the imaging cycle and increase throughput.

Also, although it is assumed in this embodiment that X-rays are generated continuously, this is not restrictive. Pulsed X-rays may be generated according to the integration interval of the two-dimensional detector 12 based on the encoder signal.

Also, the rotation start angle does not need to be set exactly to “L” or “R,” and may be set approximately to the left or right side. It may shift toward the CW or CCW direction as long as the effect of the present invention can be achieved.

Also, the present invention can be applied not only to the configuration in which imaging is performed by rotating only the human body 16, but also to a system in which imaging is performed by integrally rotating an imaging system consisting of the X-ray generator and two-dimensional detector 12 around the human body 16.

Next, detailed description will be given of the process (S101) of determining the imaging start angle (transition angle) from the rotation start angle and rotational direction in such a way as to minimize the exposure dose.

First, the exposure dose will be defined. Calculation of the exposure dose according to the present invention is based on an idea proposed by the International Commission on Radiological Protection (ICRP). The ICRP adopts the exposure dose (the unit is mSv) to assess the risk of exposure, i.e., stochastic effect on the whole body. The exposure dose is calculated using the following equation.
[Effective dose]=[equivalent dose]×[tissue weighting factor (W_{T})] Eq. (1)

where the tissue weighting factor (W_{T}) is a relative ratio of sensitivity to the stochastic effect on an organ/tissue. Table 3 shows tissue weighting factors of individual organs/tissues.

TABLE 3

| | Tissue |
|---|---|
| | Weighting Factor |
| Organ/Tissue | (W_{T}) |
| Reproductive Organs | 0.20 |
| Red Bone Marrow, Colon, | 0.12 |
| Lungs, Stomach | |
| Bladder, Breasts, Lever, | 0.05 |
| Esophagus, Thyroid Gland | |
| Skin, Bone Surfaces | 0.01 |
| Remainder | 0.05 |

The equivalent dose (the unit is mSv) in Eq. (1) represents the effect of radiation on the human body which varies with the type and energy of radiation. It is determined using Eq. (2) based on an average absorbed dose of the organ/tissue.
[Equivalent dose]=[absorbed dose]×[radiation weighting factor (W_{R})] Eq. (2)

where the radiation weighting factor (W_{R}) has been established as shown in Table 4. The absorbed dose (the unit is mGy) is the dose which results when 1 J of energy is absorbed per 1 kg and is determined for each organ/tissue.

TABLE 4

| | Radiation |
|---|---|
| Type and Energy of | Weighting Factor |
| Radiation | (W_{R}) |
| Photon (γ ray, X ray) | 1 |
| Electron (β ray) | 1 |
| Neutron E < 10 Kev | 5 |
| Photon (2 Mev < E) | 5 |
| α particles, Fission | 20 |
| Fragment, Heavy Nucleus | |

Thus, the effective dose can be found by determining the absorbed doses of organs/tissues during half scans with varied imaging start angles and performing calculations using Eqs. (2) and (1).

As described above, the half scan method described above determines the imaging start and end positions such that radiations will enter the human body from the rear during CT imaging by half scan. This makes it possible to reduce the exposure dose (effective dose) to the patient. Consequently, even if CT imaging is repeated periodically or frequently for medical examination or catamnestic observation, it is possible to reduce risks resulting from radiations.

Next, description will be given of how to determine the imaging start angle of a half scan, which is a main part of this embodiment. First, the inventors paid attention to the structure of the human body. Most of the organs which are assigned a tissue weighting factor in Table 3 are located in the front or central part of the human body. It would be right to think that the only organs located in the rear part of the human body are red bone marrow and back muscles, the latter of which are classified into the “remainder.” When the arrangement of human organs is viewed schematically, the back muscles are arranged in such a way as to guard the organs. The back muscles are classified into the “remainder” in Table 3 and assigned a small tissue weighting factor. Thus, during a half scan, the X rays incident on the human body from the rear are attenuated by the back muscles before being absorbed by organs. Since the doses reaching the detector are the same in principle regardless of whether X rays enter the human body from the front or rear, it should be advantageous in terms of exposure dose (effective dose) to direct the X rays at the human body from the rear where tissues/organs with a small tissue weighting factor are located.

To verify this hypothesis, an experiment was actually conducted using a human phantom such as described in reference 1. Table 5 shows results of the experiment. Imaging conditions for an imaging apparatus were equivalent to those used by the inventors for clinical experiments at a hospital. Specifically, the following conditions were used: an X-ray tube voltage of 120 kV, X-ray tube current of 40 mA, added filter made of copper 0.15 mm thick, 5-second scan (full scan), and 2.6-second scan (half scan). The entire area of the chest (350 mm high) was scanned. The effective energy of the X rays was 51.5 keV. In table 5, the absorbed doses (mGy) were measured in relation to a full scan from the left, a front-incident half scan, and a rear-incident half scan, which were taken twice. The front-incident half scan is a scan taken by emitting X rays in the directions “R-→A-→L” or “L→A→R” in FIG. 4. Similarly, the rear-incident half scan is a scan taken by emitting X rays in the directions “R→P→L” or “L→P→R” in FIG. 4. However, according to this embodiment, the fan angle is 7.2 degrees. Thus, the data collection angle for the half scan is actually 187.2 degrees, but assumed here to be approximately 180 degrees.

Effective doses were calculated, using Eqs. (2) and (1) and Tables 3 and 4, from absorbed doses obtained from the human phantom. The average effective dose was 0.49 mSv for the full scan, 0.30 mSv for the front-incident half scan, and 0.19 mSv for the rear-incident half scan. This means that the rear-incident half scan reduces the exposure by 35% compared to the front-incident half scan. Incidentally, the sum of the does in the front-incident half scan and rear-incident half scan equals the does in the full scan. This demonstrates the credibility of the experiment.

TABLE 5

| | | | Front- | | Rear- | |
|---|---|---|---|---|---|---|
| | | | incident | | incident | |
| | | | half scan | | half scan | |
| Examination | Chest | | Chest | | Chest | |
| Tube voltage [kV] | 120 | | 120 | | 120 | |
| Effective Energy | 51.5 | | 51.5 | | 51.5 | |
| [keV] | | | (Cu: 0.15 mm) | | | |
| Tube current [mA] | 40 | | 40 | | 40 | |
| Length of scanned | 350 | | 350 | | 350 | |
| volume [mm] | | | | | | |
| | Organ dose [mGy] | | | | | |
| Testes (male) | 0.00 | 0.00 | 0.01 | 0.00 | 0.00 | 0.00 |
| Ovaries (female) | 0.02 | 0.01 | 0.02 | 0.02 | 0.02 | 0.01 |
| Red bone marrow | 0.33 | 0.33 | 0.17 | 0.16 | 0.18 | 0.18 |
| Colon | 0.18 | 0.18 | 0.12 | 0.11 | 0.06 | 0.06 |
| Lungs | 1.01 | 1.01 | 0.59 | 0.52 | 0.46 | 0.46 |
| Stomach | 0.94 | 0.95 | 0.70 | 0.68 | 0.28 | 0.28 |
| Bladder | 0.01 | 0.01 | 0.01 | 0.01 | 0.01 | 0.01 |
| Breasts | 1.02 | 1.02 | 0.78 | 0.74 | 0.28 | 0.28 |
| Lever | 0.85 | 0.85 | 0.52 | 0.44 | 0.39 | 0.38 |
| Esophagus | 0.84 | 0.83 | 0.49 | 0.48 | 0.36 | 0.37 |
| Thyroid gland | 0.26 | 0.24 | 0.18 | 0.16 | 0.08 | 0.08 |
| Bone surfaces | 0.93 | 0.93 | 0.53 | 0.47 | 0.47 | 0.47 |
| Skin | 0.27 | 0.27 | 0.13 | 0.13 | 0.12 | 0.12 |
| Remaining | 0.56 | 0.56 | 0.35 | 0.32 | 0.24 | 0.24 |
| tissues/organs | | | | | | |
| (male) | | | | | | |
| Remaining | 0.50 | 0.50 | 0.31 | 0.28 | 0.21 | 0.21 |
| tissues/organs | | | | | | |
| (female) | | | | | | |
| Womb (female) | 0.01 | 0.01 | 0.01 | 0.01 | 0.01 | 0.01 |
| Effective dose | 0.49 | 0.48 | 0.31 | 0.29 | 0.19 | 0.19 |
| (male) [mSv] | | | | | | |
| Effective dose | 0.48 | 0.48 | 0.31 | 0.29 | 0.19 | 0.19 |
| (female) [mSv] | | | | | | |

Incidentally, in the above embodiment, an irradiation range for the half scan begins with a flank of the subject (when the face of the back rest 13 is substantially parallel to a direction of the center of the radiations from the X-ray generator 11), passes through the back (while the back rest 13 is turned to a side where the radiations enter), and ends with the opposite flank (when the face of the back rest 13 is substantially parallel to a direction of the center of the radiations from the X-ray generator 11). To implement such irradiation control, the CT imaging apparatus uses the back rest 13 having the face opposite to the back of the subject and performs control by assuming that the surface of the back rest 13 corresponds to the back of the subject. That is, for the CT imaging apparatus, the irradiation range is such that two rotational positions at which a first direction going from the center of rotation to the back rest 13 and a second direction going from the center of rotation to the X-ray generator 11 intersect approximately at right angles will be the irradiation start and end positions. Of the two rotational positions, the one located in a range in which the angle formed by the first and second directions decreases with rotation is the irradiation start position. In this way, according to the above embodiment, the back rest 13 is used as a reference which defines rotational position (rotational position of the subject), but such a reference is not limited to the back rest 13. For example, a support member may be installed to support the front face (abdomen and chest) of the human body and control may be performed by assuming that the support member corresponds to the front face of the human body. Also, a chair with a fixed sitting direction may be used alternatively.

In that case, the CT scanning apparatus can be configured as follows. Specifically, a support member can be installed in the CT scanning apparatus to support the subject while defining a backward direction going from the center of relative rotation of the subject to the back of the subject. The two rotational positions at which the backward direction of the subject and irradiation direction going from the X-ray generator 11 to the center of relative rotation intersect approximately at right angles can be set as the irradiation start and end positions. Of the two rotational positions, the one located in a range in which the angle formed by the backward direction and irradiation direction decreases with the relative rotation will be the irradiation start position.

According to the present invention, in half scan CT imaging, the start and end positions of the half scan are determined such that the human body will be irradiated from the rear. The use of this scanning method makes it possible to reduce the effective dose (exposure dose) to the patient. By reducing the exposure dose in this way, it is possible to decrease the harmful effects of radiations even if CT imaging is repeated periodically or frequently for medical examination or catamnestic observation.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2005-320007, filed Nov. 2, 2005, which is hereby incorporated by reference herein in its entirety.

What is claimed is:

1. A radiographic imaging apparatus which collects image data to perform CT half scan imaging, comprising:
a rotation unit adapted to rotate a subject relative to a radiation source and radiation detector;
an irradiation unit adapted to irradiate radiations from the radiation source to the subject rotated by the rotation unit;
a support member, rotated with the subject, which has a face to support the back of the subject; and
a control unit which controls the irradiation unit to execute the irradiation while the support member is turned to a side where the radiations enter.

2. A radiographic imaging apparatus according to claim 1, wherein the control unit controls the irradiation unit to start and finish the irradiation when the face of the support member is substantially parallel to a direction of the center of the radiations from the radiation source.

3. A radiographic imaging apparatus which collects image data to perform CT half scan imaging, comprising:
a rotation unit adapted to rotate a subject relative to a radiation source and radiation detector;
an irradiation unit adapted to irradiate radiations from the radiation source to the subject rotated by the rotation unit;
a support member, rotated with the subject, which has a face to support the front face of the subject; and
a control unit which controls the irradiation unit to execute the irradiation while the support member is not turned to a side where the radiations enter.

4. A radiographic imaging apparatus according to claim 3, wherein the control unit controls the irradiation unit to start and finish the irradiation when the face of the support member is substantially parallel to a direction of the center of the radiations from the radiation source.
